SEQUENCE LISTING (21 sequences)

SEQ ID NO: 1 - DNA, 35 bp, Artificial sequence
  PCR primer
ctgatctctacggttatgggcaattaaatgacagc

SEQ ID NO: 2 - DNA, 35 bp, Artificial sequence
  PCR primer
gctgtcatttaattgcccataaccgtagagatcag

SEQ ID NO: 3 - DNA, 29 bp, Artificial sequence
  PCR primer
ccgctcgagatgcatggagatacacctac

SEQ ID NO: 4 - DNA, 27 bp, Artificial sequence
  PCR primer
gctctagattatggtttctgagaacag

SEQ ID NO: 5 - DNA, 28 bp, Artificial sequence
  PCR primer
ggaattcgcccagaccataggagagatg

SEQ ID NO: 6 - DNA, 28 bp, Artificial sequence
  PCR primer
ccgctcgagtttacccggggacagggag

SEQ ID NO: 7 - DNA, 28 bp, Artificial sequence
  PCR primer
ggcgcgccatgcatggagatacacctac

SEQ ID NO: 8 - DNA, 27 bp, Artificial sequence
  PCR primer
ggggtacctggtttctgagaacagatg

SEQ ID NO: 9 - DNA, 26 bp, Artificial sequence
  PCR primer
ggggtaccctccagccaggggctgag

SEQ ID NO: 10 - DNA, 27 bp, Artificial sequence
  PCR primer
ccgctcgagtcatttacccggggacag

SEQ ID NO: 11 - DNA, 26 bp, Artificial sequence
  PCR primer
ggggtacccgagggtgagtactaagc

SEQ ID NO: 12 - DNA, 27 bp, Artificial sequence
  PCR primer
ccgctcgagtcatttacccggggacag

SEQ ID NO: 13 - DNA, 27 bp, Artificial sequence
  PCR primer
ccgctcgagctccagccaggggctgag

SEQ ID NO: 14 - DNA, 27 bp, Artificial sequence
  PCR primer
ccgctcgagtcatttacccggggacag

SEQ ID NO: 15 - DNA, 27 bp, Artificial sequence
  PCR primer
ccgctcgagcgagggtgagtactaagc

SEQ ID NO: 16 - DNA, 27 bp, Artificial sequence
  PCR primer
ccgctcgagtcatttacccggggacag

SEQ ID NO: 17 - protein, 10 aa, Artificial sequence
  Linking polypeptide
DDDDKGSGSG

SEQ ID NO: 18 - protein, 149 aa, Artificial sequence
  D1 domain of hHLAG-3
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASM

SEQ ID NO: 19 - protein, 90 aa, Artificial sequence
  D2 domain of hHLAG-3
TASPPGSLRASDWVILNCSFSRPDRPASVHWFRNRGQGRVPVRESPHHHLAESFLFLPQVSPMDSGPWGCILTYRDGFNVSIMYNLTVLG

SEQ ID NO: 20 - protein, 91 aa, Artificial sequence
  D3 domain of hHLAG-3
LEPPTPLTVYAGAGSRVGLPCRLPAGVGTRSFLTAKWTPPGGGPDLLVTGDNGDFTLRLEDVSQAQAGTYTCHIHLQEQQLNATVTLAIIT

SEQ ID NO: 21 - protein, 82 aa, Artificial sequence
  D4 domain of hHLAG-3
VTPKSFGSPGSLGKLLCEVTPVSGQERFVWSSLDTPSQRSFSGPWLEAQEAQLLSQPWQCQLYQGERLLGAAVYFTELSSPG

(19) United States
(12) Patent Application Publication
(10) Pub. No.: US 2008/0003235 A1
(43) Pub. Date: Jan. 3, 2008

(54) Vaccine Composition Comprising a Class II Cmh Lignd Coupled With an Antigen, Method for the Preparation and the Use Thereof

(76) Inventor: Frederic Triebel, Versailes (FR)

Correspondence Address: DAVID R PRESTON & ASSOCIATES APC, 5850 OBERLIN DRIVE, SUITE 300, SAN DIEGO, CA, 92121, US

(21) Appl. No.: 11/547,452

(22) PCT Filed: Apr. 13, 2005
(86) PCT No.: PCT/FR05/00894

§ 371 Date: Oct. 4, 2006

(30) Foreign Application Priority Data
Apr. 13, 2004 (FR) 04/03848

(51) Int. Cl. A61K 39/385; A61P 35/00; A61P 37/00; C07K 14/705

(52) U.S. Cl. 424/192.1; 424/193.1; 424/196.11; 424/197.11; 530/300

(57) ABSTRACT

The invention relates to a coupling product consisting of a first type of antigen protein and a second type of ligand protein for class II of MHC, wherein said two protein types are coupled by one or several stable bonds in biological media.

This application is the United States National Phase application of, and claims benefit of priority to, International Patent Application No. PCT/FR2005/000894, filed on Apr. 13, 2005, which claims priority to French Patent Application No. 04/03848, filed Apr. 13, 2004 both of which applications are incorporated herein by reference.

The present invention pertains to the field of therapeutic vaccines. The invention relates to a vaccine able to enhance the immunogenic action of an antigen via the coupling of this antigen to a class II MHC ligand, such as LAG-3 or CD4. The invention concerns very particularly a coupling product, particularly in the form of a fusion protein, comprising at least one antigen specific for the disorder against which the induction of immunisation is desired and at least one class II MHC ligand.

The natural ligands of the class II MHC proteins, such as LAG-3, also designated as CD223, or CD4 are involved in immune recognition, specially at the level of the interaction with various lymphoid cells such as lymphocytes and antigen-presenting cells.

It was proposed in the PCT application WO 99/04810 to use a class II MHC ligand, such as LAG-3, as an adjunct for the manufacture of vaccines for cancer immunotherapy.

The studies conducted within the context of the present invention have now allowed the remarkable efficacy of immunisation by a fusion protein consisting of LAG-3 and an antigen to be demonstrated. Indeed, the applicant observed a very marked CD8 response obtained with very low doses of a LAG-3-antigen coupling product in vitro, compared to the doses used in the former art for vaccine compositions comprising an antigen and the LAG-3 protein as an adjunct.

Such efficacy results from the addition of the conventional LAG-3Ig adjunct effect to a targeting effect of the antigen (vectorisation) on the presenting cells (dendritic cells), which allows the internalisation of the LAG-3-bound antigen to the class II MHC molecules. An important “cross-presentation” phenomenon of the antigen towards the route of presentation of the class I MHC therefore allows the induction of T-cell CD8 responses, whereas only CD4 responses are expected with an exogenous antigen such as a vaccinal protein.

Furthermore, the experimental data reported below show the rapid internalisation at 37° C. of the LAG-3Ig/Ag coupling product by confocal microscopy (internalisation within 15 minutes).

Therefore, an object of the present invention is a coupling product, preferably of a substantially proteinaceous nature, consisting of a first class of antigen-type proteins and a second class of proteins of the class II MHC ligand type, both classes of proteins being coupled by one or several bonds which are stable in biological environments.

By a bond which is stable in biological environments, it is meant, but without limitations, covalent bonds (for example, amide bonds or disulphide bonds), ionic, hydrogen, of Van der Waals, hydrophobic and any combination thereof, said bond allowing the integrity of the coupling product according to the invention in biological environments to be maintained.

Preferably, both classes of proteins of the coupling product according to the invention are bound by hydrogen links or covalent bonds and in a particularly preferred manner by covalent bonds.

According to a first embodiment, a coupling product of the invention is characterised by the fact that both classes of proteins are bound by hydrogen bonds.

According to a second embodiment, a coupling product of the invention is characterised by the fact that both classes of proteins are linked by covalent bonds. Both classes of proteins may be bound by covalent bonds, either directly or indirectly via a linker or a linking molecule.

A direct covalent bond is defined as the pooling of one or several electrons of the atoms of the first class of proteins and the second class of proteins according to the invention.

Examples of linkers or linking molecules include a polypeptide or an amino acid, a polysaccharide or a monosaccharide, a polynucleotide or a nucleic acid, an alkyl, cyclo-alkyl or aryl group.

An advantageous embodiment of the invention is a coupling product which is in the form of a fusion protein in which both classes of proteins are directly or indirectly linked by one or several peptide bonds. By fusion protein, it is intended coupling product allowing maturation and expression of both classes of proteins according to the invention in a single and same reading phase. In the case of fusion proteins, the antigen(s) and ligand(s) of class II MHC are linked by covalent bonds at the N and/or C termini.

Thus, different combinations of recombinant proteins were prepared in which hLAG-3 was used as a MHC ligand in its 4-domain Ig (D1D4) or 2-domain Ig (D1D2) form and in which the viral antigens E7 or gag-nef used as an antigen were placed either at the N— or C-terminal end of hLAG-3Ig.

As examples of coupling products in which the first and second class of proteins are linked by covalent bonds, one may mention those of the following formula (I):
[(Ag)ₙ(X)ₘ(Y)ₚ]_{q}

in which:

Ag represents the antigen of the first class of protein and n represents the number of antigen molecules in the coupling product, n being an integer from 1 to 5,

Y represents a class II MHC ligand of the second class of proteins and p represents the number of class II MHC ligand in the coupling product, p being an integer from 1 to 2,

X represents the bond between Ag and Y and m represents the number of bonds between Ag and Y in the coupling product, m being an integer from 1 to 5,

q is an integer from 1 to 5.

When both classes of proteins are linked directly or indirectly by covalent bonds, X is chosen in the group comprising a covalent bond, a linker or a linking molecule, as defined above.

Therefore, in a coupling product according to the invention, the first class of proteins may comprise a single antigen or several different or identical antigens. Preferably, the coupling product according to the invention comprises a single antigen.

Likewise, in a coupling product according to the invention, the second class of proteins may comprise a single class II MHC ligand or several identical or different class II MHC ligands. Preferably, the coupling product according to the invention comprises a single class II MHC ligand.

When the coupling product according to the invention comprises several antigens and/or class II MHC ligands:
each class of proteins may be grouped together in the form of a polymer, for example a dimer of class II MHC ligands, linked by covalent bonds to a monomer or polymer of the antigen,
the antigens and/or class II MHC ligands are alternated and may form polymers of repeated units.

The protein constructs above may be prepared in the form of fusion protein by using any method well-known to those skilled in the art such as the recombinant DNA technique or chemical synthesis. The recombinant DNA technique is based on the preparation of recombinant DNAs comprising the nucleotide sequences encoding the protein constructs according to the invention.

In the case of preparation of the coupling products of the invention by chemical synthesis, the antigen(s) and class II MHC ligand(s) may also be linked by covalent bonds at one or several amino acid side chains. In the case of binding via amino acid side chains, the coupling products must retain the property of binding to the class II MHC of the dendritic cells with a high level of affinity and internalise the antigen.

According to a preferred embodiment of the invention, in the coupling products according to the invention, the second class of proteins is chosen from the group comprising hLAG-3, its homologues, fragments and derivatives and the mixtures thereof.

The homologues, fragments and derivatives of LAG-3 are those which are able to assure high affinity binding to the class II MHC of the dendritic cells and internalisation of the antigen of the first class of protein.

Homologue is intended to mean a protein LAG-3 from a species other than humans, for example murine LAG-3 (mLAG-3). Advantageously, the protein sequence has at least 70% homology with the protein sequence of human LAG-3, preferably of at least 80% and more preferably, of at least 90%. The homology between two protein sequences corresponds to the percentage of identical amino acids localised at an identical or similar position in the two protein chains. The percentage of homology is calculated using a BLAST algorithm available at the site of the NCBI (National Center for Biotechnology Information; http://www.ncbi.nlm.nih.go/) using the BLOSUM 62 matrix.

Lag-3 fragments are defined as protein sequences of LAG-3 able to assure high affinity binding to the class II MHC of the dendritic cells and internalisation of the antigen of the first class of protein, the aforementioned protein sequences of which having a length between 50 and 200 amino acids, preferably between 60 and 175 amino acids and more preferably between 75 and 160 amino acids.

As an example of hLAG-3 fragments may be mentioned in particular the soluble fractions including at least the two Ig type extracellular N-terminal domains. These domains are described in particular in WO 91/10862 and WO 95/30750. The invention encompasses specifically in the coupling product a LAG-3 fragment chosen from the group comprising the D1-D2 (SEQ ID No. 18 and SEQ ID No. 19) and D1-D4 (SEQ ID No. 18 and SEQ ID No. 19, SEQ ID No. 20 and SEQ ID No. 21) fragments. Therefore, in the examples reported in the experimental part, hLAG-3 was used in its 4-domain (D1D4) or 2-domain (D1D2) form, the two domains D1-D2 being sufficient in order to guarantee high affinity binding to the class II MHC of the dendritic cells followed by internalisation.

LAG-3 derivatives or fragments thereof below comprise those the amino acid sequence of which has been modified by deletion, addition or substitution of one or several amino acids, the aforementioned derivatives being able to assure high affinity binding to the class II MHC of the dendritic cells and internalisation of the antigen of the first class of protein. May be mentioned for example the substitution of one or several arginines at position 73, 75 and/or 76 by glutamic acid, as described in the international patent applications WO 95/30750 and WO 98/23741. It may also be a splicing variant of LAG-3 as described in the international patent application WO 98/58059.

As an example of a binding test allowing to determine whether a homologue, fragment of derivative of LAG-3 falls within the framework of the invention, one may mention:
indirect immunofluorescence cell marking, using a B line transformed by EBV expressing class II MHC molecules. A positive control is always used (pan-class II antibody designated 9.49 or I3 followed by a GAM-FITC). Saturation of marker is reached at 30 μg/mL or 10 μg/mL of LAG-3Ig (first layer) developed with a GAH-FITC (second layer). A signal is always detected at 3 or 1 μg/mL of LAG-3Ig. These results must be obtained with a fusion protein LAG-3Ig/Ag;
Bioacore type marking in which a class II MHC protein is bound to a slide and the affinity of the binding of LAG-3Ig or LAG-Ig/Ag is measured. The two types of “binding” must have similar Kd values (dissociation constant) and in any case less than 5×10⁻⁸ and preferably less than 3×10⁻⁹.

As an example of an internalisation test allowing to determine whether a homologue, fragment or derivative of LAG-3 falls within the framework of the invention, one may mention marking on a slide and analysis with a confocal microscope. Dendritic cells are obtained within 6 days from purified human monocytes (in vitro culture with IL-4 and GM-CSF) according to the following procedure:
collection of the cells and counting;
centrifugation for 5 minutes at 1100 rpm;
dilution of cells at 1 million/mL in PBS 1X (following equilibration at 37° C.);
plating 300 μL of cell suspension/slide and leaving cells to adhere on the slides coated with polylysine for 30 min at 37° C.;
removal of the fluid and saturation with 500 μL of PBS 1X/0.1% NaN₃/3% milk for 30 min. at 37° C.;
putting the slides on ice (4° C.) and cooling for 10-15 minutes;
removal of the fluid and gentle washing with cold PBS 1X;
plating of 400 μL of the antibody (or hLAG-3Ig protein at 30 μg/mL) diluted in PBS 1X/0.1% NaN₃/3% cold milk and incubation at 4° C. for 30 min;
removal of the fluid and gentle washing with cold PBS 1X;
repetition of the 2 last steps for each additional marking (GAH-FITC for the LAG-3Ig marking);
leaving the slides at 37° C. for 15-20 min. (in the incubator in order to allow internalisation);
plating of 400 μL of cold PBH 1X/2% cold PFA and fixation of cells for 15 min. at 4° C.;
removal of the fluid and gentle washing with cold PBS 1X;
addition of 20-30 μL of Fluoromount G, placing the cover slide with caution and leaving to dry at room temperature during 1-2 hours before placing at 4° C.;
analysis of the slides by confocal microscopy.

According to a preferred embodiment of the invention, in the coupling products according to the invention, the first class of antigen-type protein is chosen from the group comprising antigens specific of a disorder, the treatment of which requires a T-cell response. More specifically, the group comprising viral antigens, bacterial antigens, tumoral antigens mentioned, parasite antigens and mixtures thereof may be.

Thus, the first class of antigen-type protein is a viral antigen, preferably chosen from the group comprising the viruses HPV, HBV, HCV, HIV, EBV, CMV and their mixtures, and very particularly, the group comprising the HPV E7 antigen and the HIV gag-nef antigen. Indeed, with the aim of developing new vaccinal proteins, LAG-3 was fused with viral antigens (E7 from HPV-16 or gag-nef from HIV-1). The aim was to obtain, in addition to the adjunctive (immunostimulant) effect of LAG-3, targeting of the antigen to immature dendritic cells. These cells express the class II MHC molecules, the LAG-3 ligands, which are very rapidly recycled towards the inside of the cell, thereby pulling with it the antigen coupled to LAG-3. Fusion molecules comprising hLAG-3Ig and the viral antigens were constructed in this way, expressed in mammalian cells, purified and tested functionally.

The first class of antigen-type protein may also be a bacterial antigen chosen from the group of intracellular bacteria of tuberculosis, leprosy and listeria. It may furthermore advantageously be a tumoral antigen selected from the group including CEA, Melan A, PSA, MAGE-3, HER2/neu, E6 and E7 protein from HPV (cancer of the cervix).

The invention provides a new vaccinal strategy based on the use of LAG-3, which is based on the use of a natural ligand and not an antibody. This offers the advantage of allowing a very marked reduction in the doses of LAG-3Ig and antigen injected, which in turn allows easier development on an industrial scale of therapeutic vaccine compositions containing them. Furthermore, these compositions allow high T-cell CD8 responses to be induced in humans with low doses of therapeutic vaccines. One must emphasise the value of the demonstrated increase in CD4 responses which will support the CD8 response in vivo and allow the latter to be very high and prolonged, i.e. effective in destroying reservoirs of virus or tumour cells.

Thus, the invention also concerns a vaccine composition comprising at least one coupling product such as defined above, advantageously combined with a pharmaceutical vehicle in a form allowing oral, cutaneous, subcutaneous, topical, intramuscular, intravenous or intra-arterial administration or administration in any liquidian compartment of the body.

Advantageously, the compositions of the invention contain between 0.1 μg/mL and 1 mg/mL, preferably between 0.1 μg/mL and 100 μg/mL, more preferably between 0.1 μg/mL and 10 μg/mL and particularly preferably between 0.1 μg/mL and 1 μg/mL of coupling product. These compositions are assayed by ELISA.

The invention also refers to a vaccination method of an individual consisting in administering to an individual suffering from a disorder corresponding to the antigen of the first class of protein, a sufficient quantity of a composition such as defined above.

The invention furthermore concerns the use of a coupling product such as defined above as a drug.

Advantageously, the invention concerns the use of a coupling product such as defined above for the preparation of an immunogenic composition capable of inducing immunisation, preferably capable of inducing a specific T-cell CD4 and/or CD8 response.

Indeed, the data reported in the experimental part below show that coupling of two different proteins (E7 and gag-nef) at the C-terminal position of the protein LAG-3Ig achieves a very high level of immunisation in vitro, both with regard to the T CD4 responses (presentation by the class II MHC molecules) and CD8 (presentation by the class I MHC molecules). This in vitro immunogenicity was defined with PBMCs of healthy donors.

The CD4 responses were studied using Elispot by quantifying the cells secreting intracellular γ-interferon in response to a 48-day exposition to the E7 or gag-nef antigen presented in the form of a protein, which is therefore taken up by the dendritic cells of the PBMCs and presented by the class II MHC molecules in the form of peptides of 11 to 20 amino acids.

The CD8 responses were studied using Elispot by quantifying the cells secreting γ-interferon in response to a 48-day stimulation by peptides. 9 to 10 amino acids long presented by the class I MHC molecules.

In both cases, prior amplification of the T CD4 or CD8 responses was obtained after three in vitro stimulations with the antigen.

These high CD4 and CD8 responses using Elispot (due to priming of naive T cells with healthy HPV-16⁻ and HIV⁻ volunteers or boosting with healthy HPV-16⁺ or HIV⁺ volunteers) are not obtained when the E7 or gag-nef antigen is added alone and when a mixture of LAG-3Ig and E7 or LAG-3Ig and gag-nef is added at lower levels.

Advantageously still, the invention also refers to the use of a coupling product according to the invention for the manufacture of a drug intended for treating infectious diseases and/or cancer. As an example of infectious diseases, viral, bacterial and parasitic infections may be mentioned. Preferably, the treatment of infectious diseases or cancer implies an immune response via the T CD8+ cells.

According to a particular embodiment of the invention, the coupling product according to the invention is used in order to manufacture a drug intended for treating infectious diseases and/or cancer in which the second class of proteins of the class II MHC ligand type according to the invention is capable of inducing a antigen-specific immune response via the T cells.

Other advantages and features of the invention will become apparent from the following examples taken jointly with the appended drawings, among which:

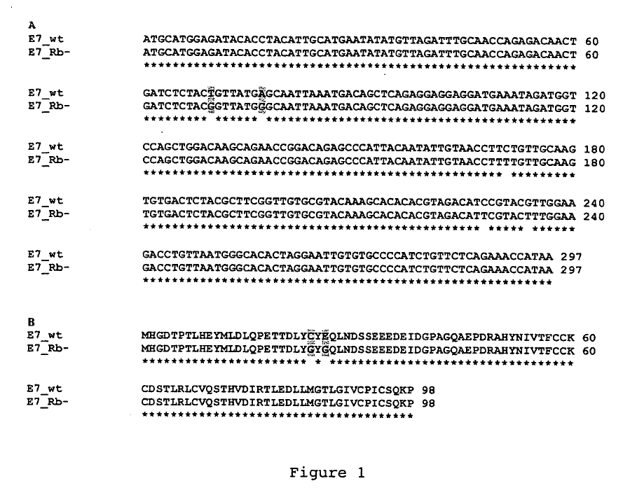

FIG. 1 shows the nucleic (A) and peptide (B) sequence alignment between the theoretical sequence of E7wt and that of E7Rb obtained after sequencing following site-directed mutagenesis (2 point mutations)

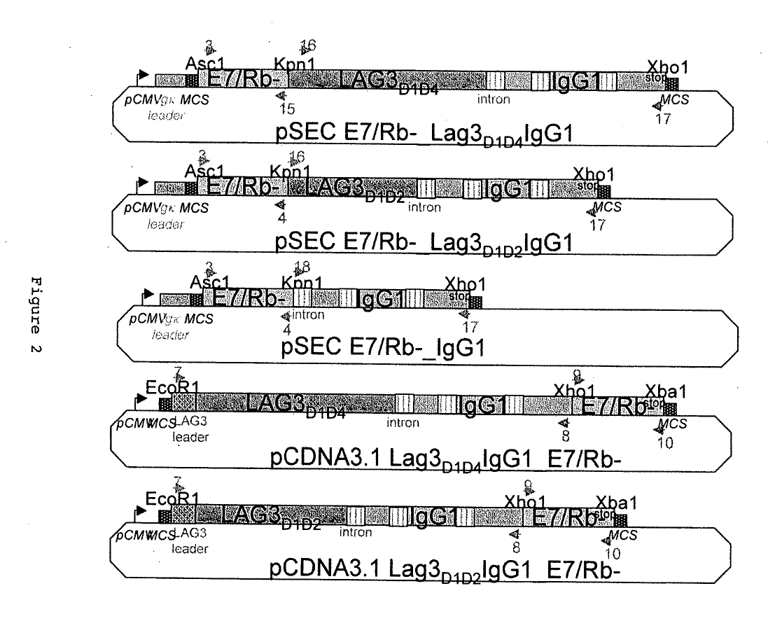

FIG. 2 summarises the cloning strategy for the expression vectors pCDNA3 and pSEC expressing the recombinant proteins hLAG-3Ig-E7, E7-hLAG-3Ig and E7Rb⁻Ig as a control. The oligonucleotides used are represented by arrows.

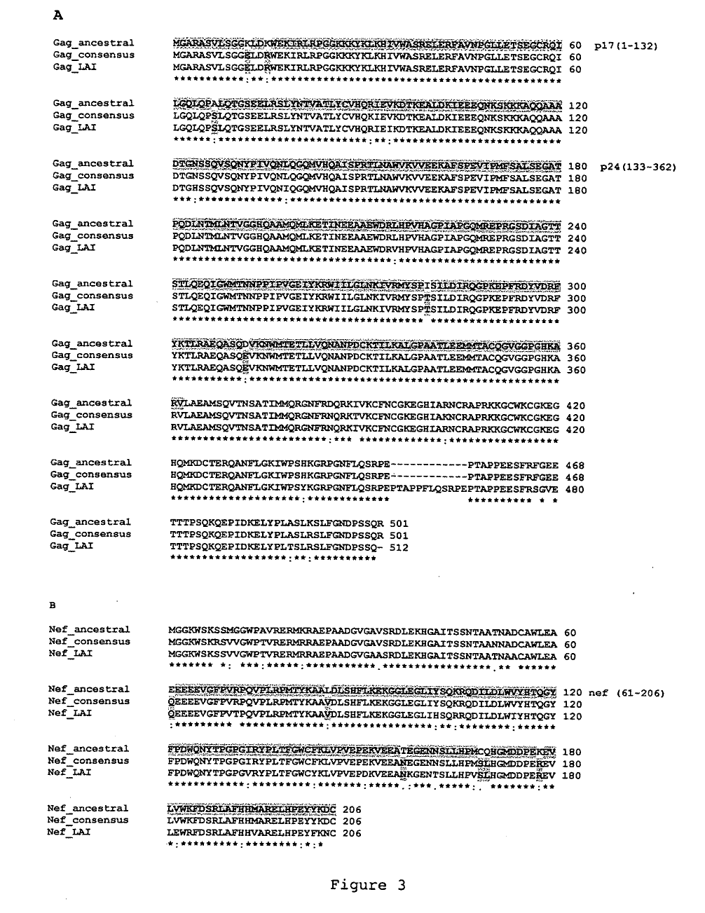

FIG. 3 shows the sequence alignment between the ancestral group B, consensus group B and LAI sequences, for the gag (panel A) and nef (panel B) proteins. The amino acids chosen for p17, p24 and nef in order to optimise the gag-nef protein are underlined.

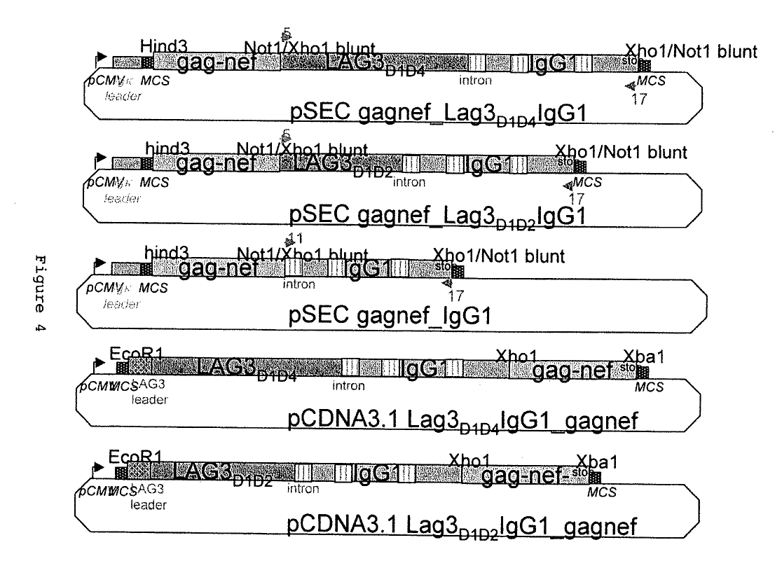

FIG. 4 summarises the cloning strategy of the expression vectors pCDNA3 and pSEC expressing the recombinant proteins hLAG-3Ig-gagnef, gagnef-hLAG-3Ig, and gagnef-Ig as a control. The oligonucleotides used are represented by arrows.

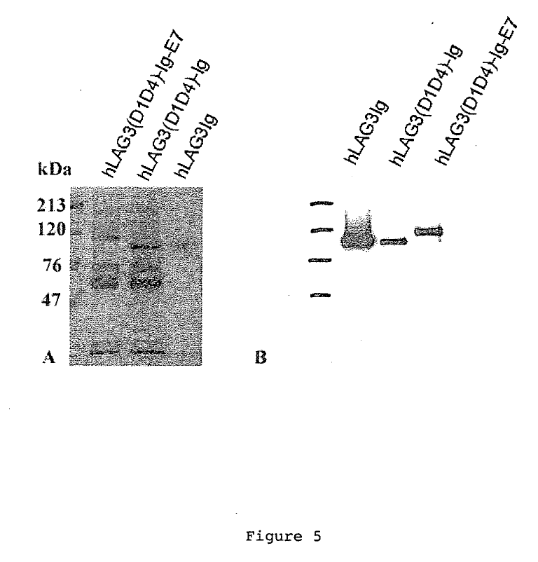

FIG. 5 shows the SDS-PAGE gels of the purified hLAG-3_{(D1D4)}Ig and hLAG-3_{(D1D4)}Ig-E7 proteins; the purified hLAG-3Ig protein (at 0.71 mg/mL) was also deposited as a reference. The molecular weight marker is the same for the three gels (Biorad high range marker). A: Coomassie blue staining. B: anti-hLAG-3 by Western blot with 17B4 monoclonal antibody.

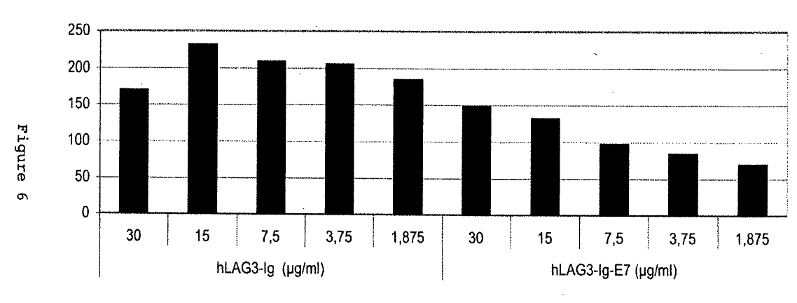

FIG. 6 shows the binding of hLAG-3_{(D1D4)}Ig and hLAG-3_{(D1D4)}Ig-E⁷ to the class II MHC of EBV-transformed B cells (expressed as the mean fluorescence weighted by the percentage of positive cells: ordinate axis).

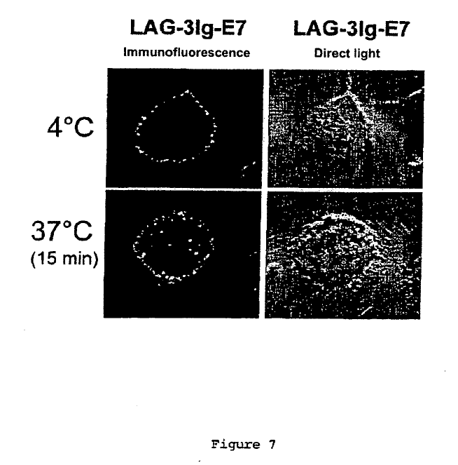

FIG. 7 shows the internalisation of hLAG-3_{(D1D4)}Ig-E7 in immature human dendritic cells at 37° C., detected by immunofluorescence (points inside the cells).

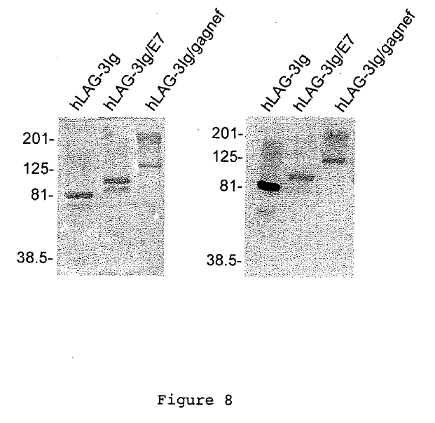

FIG. 8 shows the SDS-PAGE gels of purified hLAG-3_{(D1D4)}Ig/E⁷ and hLAG-3_{(D1D4)}Ig/gagnef proteins; the protein hLAG-3Ig (batch PDC12.096) was also deposited as a reference (Biorad Kaleidoscope molecular weight marker). A: Coomassie blue staining. B: anti-hLAG-3 Western blot with 17B4 monoclonal antibody.

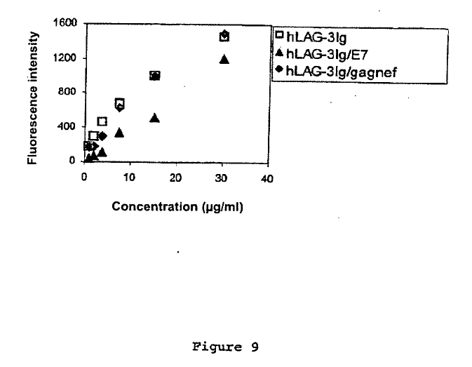

FIG. 9 shows the binding of hLAG-3Ig (batch Hénogen S017/LPC/041008), hLAG-3_{(D1D4)}Ig/E7 and hLAG-3_{(D1D4)}Ig/gagnef on the class II MHC of EBV-transformed B cells (expressed as the mean fluorescence intensity relative to concentration).

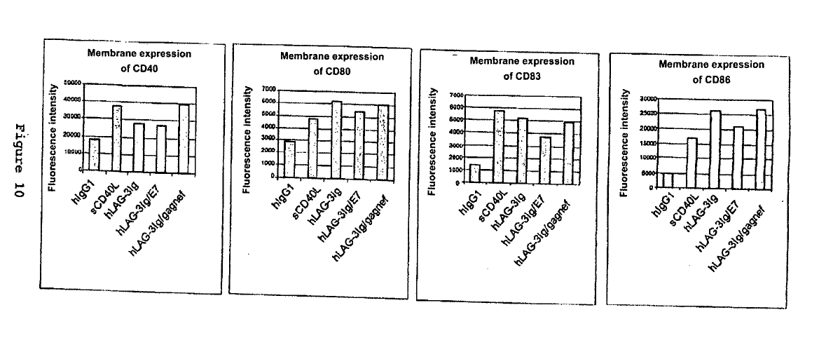

FIG. 10 shows the expression of the CD40, CD80, CD83 and CD86 activation markers at the surface of dendritic cells incubated for 2 days with human IgG1, sCD40L, hLAG-3Ig (batch Hénogen S017/LPC/041008), hLAG-3_{(D1D4)}Ig/E7 or hLAG-3_{(D1D4)}Ig/gagnef. The membrane expression is proportional to the fluorescence intensity.

### 1) CONSTRUCTION OF THE EXPRESSION VECTORS

Vectors allowing expression and secretion by mammalian cells of the different recombinant proteins were constructed.

### 1.1) Vectors Used

### 1.1.1) Cloning Vectors

Two expression vectors were chosen for expressing the recombinant proteins in CHO-K1 cells:
pCDNA3.1 (+) from Invitrogen was chosen for expressing the hLAG-3Ig/antigen fusion proteins. These recombinant proteins contained at the N-terminal end the hLAG-3 leader sequence, thus allowing its secretion in the culture medium.
pSEC-tag2-hygroA and pSEC-tag2-hygroB from Invitrogen were chosen for expressing the viral antigen/hLAG-3Ig fusion proteins. This vector contained IgK leader upstream from the promoter allowing secretion of the protein.
Dα-LAG3-DID4ΔEK-hIgG1 Fusion from Henogen was chosen for expressing the hLAG-3_{(D1D4)}Ig/E⁷ and hLAG-3_{(D1D4)}Ig/gagnef fusion proteins. This vector contained a sequence coding for hLAG-3_{(D1D4)}Ig in which intron A, located upstream from the “Ig-hinge-Fc” region was replaced by a linker sequence (coding for DDDDKGSGSG, SEQ ID No. 17) in order to rule out splicing ambiguities. This vector also contained the dhfr gene allowing the cells having integrated the plasmid sequence into their genome to be selected, as well as this sequence to be amplified in the presence of methotrexate.

### 1.1.2) Source of hLAG-3Ig

Verification and Amplification of the Starting Plasmids:

hLAG-3_{(D1D4)}Ig and hLAG-3_{(D1D2)}Ig were amplified from the pCDNA3-hLAG3_{(D1D4)-}IgG1 and pCDM7 -hLAG-3_{(D1D2)}IgG1 vectors, respectively.

pDNA3-hLAG-3_{(D1D4)-}IgG1: the hLAG-3Ig insert was sub-cloned at XbaI in pCDNA3 from pCDM7-hLAG-3Ig.

These plasmids were reamplified from stocks solutions of transformed bacteria stored in glycerol at −80° C. Digestion by different restriction enzymes allowed the nature of the plasmids to be confirmed.

Sequencing of hLAG-3_{(D1D4)}IgG1 and hLAG-3_{(D1D2)}IgG1:

Since the sequences of hLAG-3_{(D1D4)}IgG1 and hLAG-3_{(D1D2)}IgG1 were still incompletely known at the beginning of the project, they were entirely sequenced after sub-cloning in pCDNA3.1+.

Thus, all remaining uncertainties concerning the number of introns in IgG1 (all the introns are present) and the splicing sequence between hLAG-3_{(D1D2)} and IgG1 were resolved.

Three mutations including two amino acid changes in the CH3 region of IgG1 were also detected, in addition to the insertion of a T at position +4 of the IgG1 A intron.

Based on these results, sequences named reconstituted hLAG-3_{(D1D4)}IgG1 and reconstituted hLAG-3_{(D1D2)}IgG1 were compiled.

### 1.2) Fusion of hLAG-3Ig and HPV-16 E7

A mutated form of E7, non-oncogenic, was cloned at the C— or N-terminal end of hLAG-3Ig (D1D4 or D1D2), in the expression vectors described above. E7 and IgG1 without LAG-3 were also fused as a control.

### 1.2.1) Mutagenesis of E7

HPV-16 E7 was double mutated in order to prevent its dimerisation with the cellular protein Rb responsible for the oncogenic activity of E7 (see Lee at al. Nature 1998 vol 391 p 859; Burg et al. Vaccine 2001 vol 19 p 3652; Boursnell et al. Vaccine vol. 14 p. 1485).

A plasmid pEF6-E7, containing the wild form of E7 (cloned at T/A) was obtained.

E7 was submitted to site-directed mutagenesis using the Quickchange kit from Stratagen in order to substitute the amino acids C₂₄ with G and E₂₆ with G. The plasmid pEF6-E7 was amplified by PCR with the complementary pair of oligonucleotides containing the desired mutations:

Oligo 1, E7 mut 5′:

| | |
|---|---|
| CTGATCTCTACGGTTATGGGCAATTAAATGACAGC | (SEQ ID NO. 1) |

Oligo 2, E7 mut 3′:

| | |
|---|---|
| GCTGTCATTTAATTGCCCATAACCGTAGAGATCAG | (SEQ ID No. 2) |

The PCR product was subsequently incubated with Dpn1, an enzyme solely active against the methylated sites, therefore digesting the template DNA on the PCR products. The obtained product was subsequently transformed into XL1-Blue bacteria.

The plasmid thus obtained was verified by digesting and sequencing of the E7mutRb— insert. The result of the sequencing indicates that the desired mutations are indeed present. Three other mutations not affecting the amino acid composition were also detected in relation to the theoretical sequence of E7 shown in FIG. 1.

### 1.2.2) Cloning of hLAG-3Ig-E7 and E7-hLAG-3Ig in the Expression Vectors

The E7/Rb— (herein below designated. E7 for simplification) and hLAG-3Ig inserts were amplified by PCR, with oligonucleotides allowing a restriction site to be added at its ends.

The high reliability enzyme Pfu turbo from Stratagene was used for the PCR procedures.

The cloning strategy is summarised in FIG. 2.

Cloning of hLAG-3Ig-E7 into pCDNA3.1:

E7 was amplified from pEF6_E7/rb— with the following pair of oligonucleotides:

Oligo 9 (E7 5′-Xho1):

| | |
|---|---|
| CCGCTCGAGATGCATGGAGATACACCTAC | (SEQ ID No. 3) |

containing a Xho1 site and the ATG of E7

Oligo 10 (E7 3′-stopXbal):

| | |
|---|---|
| GCTCTAGATTATGGTTTCTGAGACAG | (SEQ ID No. 4) |

Containing the 3′ region of E7 with the stop codon and a XbaI site.

The PCR product was digested with XhoI and XbaI before purification.

LAG-3_{(D1D2)}IgG1 and LAG-3_{(D1D4)}IgG1 were amplified from pCDM7-LAG-3_{(D1D2)*-}IgG1 and pCDN3-LAG-3_{(D1D4)-}IgG1 respectively using the following pair of oligonucleotides:

Oligo 7 (Lag3 5′-atgEcoRI):

| | |
|---|---|
| GGAATTCGCCCAGACCATAGGAGAGATG | (SEQ ID No. 5) |

containing a EcoRI site, the ATG of hLAG-3, with the secretory signal peptide,

Oligo 8 (IgG1 3′-XhoI):

| | |
|---|---|
| CCGCTCGAGTTTACCCGGGGACAGGGAG | (SEQ ID No. 6) |

containing the 3′ region of IgG1, without the stop codon.

The PCR product was digested with EcoRI and XhoI before purification.

Insertion of hLAG-3_{(D1D4)}Ig-E7 into pCDNA3.1+ was performed in two steps. Firstly, hLAG-3_{(D1D4)}Ig was ligated into pCDNA3.1+ digested with EcoRI and XhoI. The cloning intermediate pCDNA-hLAG-3_{(D1D4)}Ig was thus obtained. Subsequently, E7 was inserted into pCDNA-hLAG-3_{(D1D4)}Ig previously digested with XhoI and XbaI.

For cloning the hLAG-3_{(D1D4)}Ig-E7 fusion product into pCDNA3.1+, both inserts were first ligated together. The hLAG-3_{(D1D4)}Ig-E7 fragment thus obtained was purified on gel and subsequently inserted directly into pCDNA3.1+ previously digested with EcoRI and XbaI.

A pCDNA-hLAG-3_{(D1D2)}Ig plasmid was also prepared by ligating hLAG-3_{(D1D2)}Ig into pCDNA3.1+ digested with EcoRI and XhoI.

The inserts of these expression vectors were sequenced. The results of the sequencing confirm the correct insertion of the inserts in phase with the reading frame.

Cloning of E7-hLAG-3Ig into pSECtag-hygroA:

E7 was amplified from pEF6-E7/Rb— with the following pair of oligonucleotides:

Oligo 3 (E7 5′-AscI):

| | |
|---|---|
| GGCGCGCCATGCATGGAGATACACCTAC | (SEQ ID No. 7) |

containing a AscI site and the ATG of E7,

oligo 15 (E7 3′-Kpn1):

| | |
|---|---|
| GGGGTACCTGGTTTCTGAGAACAGATG | (SEQ ID No. 8) |

containing the 3′ region of E7 without the stop codon and a KpnI site.

The PCR product was digested with AscI and KpnI before purification.

LAG-3_{(D1D2)}IgG1 and LAG-3_{(D1D4)}Ig were amplified from pCDM7-LAG-_{(D1D2)-}IgG1 and pCDNA3-LAG-3_{(D1D4)-}IgG1, respectively, with the following pair of oligonucleotides:

Oligo 16 (Lag3 5′ (-D1KpnI):

| | |
|---|---|
| GGGGTACCCTCCAGCCAGGGGCTGAG | (SEQ ID No. 9) |

containing a KpnI site and the 5′ region of domain 1, without the signal peptide,

oligo 17 (IgGI 3′-stopXhol):

| | |
|---|---|
| CCGCTCGAGTCATTTACCCGGGGACAG | (SEQ ID No. 10) |

containing the 3′ region of IgG1 with the stop codon and a XhoI site.

The PCR product was digested with KpnI and XhoI before purification.

IgGI was amplified from pCDNA-LAG-3_{(D1D4)-}IgG1 (for cloning into pSEC at the 3′ end of E7 for use as a control without Lag3) with the following pair of oligonucleotides:

Oligo 18 (IgG1 5′ Kpn1):

| | |
|---|---|
| GGGGTACCCGAGGGTGAGYACTAAGC | (SEQ ID No. 11) |

containing a KpnI site and the 5′ region of the A intron of IgG1,

oligo 19 (IgGI 3′-stopXhoI):

| | |
|---|---|
| CCGCTCGAGTCATTTACCCGGGGACAG | (SEQ ID No. 12) |

containing the 3′ region of IgGI with the stop codon and a XhoI site.

The PCR product was digested with KpnI and XhoI before purification.

Insertion of the E7-hLAG-3Ig fusion product into pSECtag-hygroA was performed in two steps:
the digested PCR product of E7 was ligated into pSECB previously digested with AscI and KpnI. A cloning intermediate designated pSEC-E7 was thus obtained;
the PCR products of hLAG-3_{(D1D4)}Ig, hLAG-3_{(D1D2)}Ig and IgGI were subsequently ligated into pSEC-E7 previously digested with KpnI and XhoI.

These expression vectors were verified by enzymatic restriction.

Cloning of E7 into Dα-LAG3-D1D4-ΔEK-hIgG1 Fusion:

The destination vector is the vector Dα-LAG3-D1D4-ΔEK-hIgG1 Fusion from Henogen. In this vector, the coding sequence for LAG-3_{(D1D4)}Ig is inserted between the two XhoI restriction sites. As mentioned above, this vector contains the coding sequence for LAG-3_{(D1D4)}Ig in which the A intron (intron at position 5′ from the Ig hinge region) was replaced by a linker sequence (coding for DDDDKGSGSG: SEQ ID No. 17). This coding sequence for LAG-3_{(D1D4)}Ig without A intron was designated LAG-3_{(D1D4)}Ig/and by extension the same notation will be retained for the protein coded by these constructs.

The fragment encoding E7 is derived from the plasmid pcDNA3-LAG-3_{(D1D4)}-E7.

Cloning of E7 into Dα-LAG3-D1D4-ΔEK-hIgG1 Fusion was performed in two steps:

In the first step, the LAG-3_{(D1D4)}-Ig/fragment was inserted into pCDNA3-LAG-3_{(D1D4)}Ig-E^{7.}

The Dα-LAG3-D1D4-ΔEK-hIgG1 vector was cleaved by XhoI and its sticky ends were blunted with the enzyme T4 polymerase. The fragment corresponding to the Da vector without the XhoI blunt/XhoI blunt insert was retained as the final destination vector. The XhoI blunt/XhoI blunt insert was digested with the enzyme BsrGI at the level of the C intron of the sequence coding for Ig in order to eliminate the last 300 base pairs of the sequence coding for Ig which includes the stop codon (LAG-3_{(D1D4)}Ig/XhoI blunt/BsrGI insert).

The pCDNA3-LAG-3_{(D1D4)}Ig-E7 vector was digested with the enzyme EcoRI (EcoRI site contained in the Multiple cloning site of pCDNA3 upstream from the cloning site) and the sticky ends were blunted using T4 polymerase. The thus linearised plasmid was cleaved with the enzyme BsrGI in order to eliminate the sequence coding for LAG-3_{(D1D4)} and the 5′ sequence coding for Ig in order to retain the last 300 base pairs coding for Ig (EcoRI blunt/BsrGI Ig-E7 pcDNA).

The LAG-3_{(D1D4)}Ig/XhoI blunt/BsrGI insert was ligated into EcoRI blunt/BsrGI Ig-E7 pCDNA3.

Following restriction analysis of the obtained clones, a clone containing pCDNA3-LAG-3_{(D1D4)}Ig/E7 was selected.

In the second step, the LAG-3_{(D1D4)}Ig/E7 construct was cloned into Dα.

The LAG-3_{(D1D4)}Ig/E7 insert contained in pCDNA3 was cleaved by the enzyme PmeI (2 PmeI sites surrounding the Multiple Cloning Site of pCNDA3, blunt end) and ligated into Dα without the insert previously prepared by cleaving XhoI blunt/XhoI blunt and dephosphorylated.

The clones comprising the LAG-3_{(D1D4)}Ig/E7 insert in Da in the correct sense were selected by restriction analysis.

The DNA of one of the Dα-LAG-3_{(D1D4)}Ig/E7 clones was retransformed into DH5α strain and prepared by maxiprep Endofree (Qiagen) and used for transient transfection into CHO-K1 cells in order to verify the translation product of the plasmid. The original Dα-LAG3-D1D4-ΔEK-hIgG1 Fusion and pCDNA3-LAG-3_{(D1D4)}Ig vectors were used as positive controls and the Dα-vector, from which the insert was eliminated by XhoI followed by ligation, was used as a negative control. Twenty-four hours after transfection, LAG-3 was quantified in the supernatants by specific ELISA. In parallel, the recombinant proteins present in the supernatants and the cell lysates were precipitated by A-sepharose protein (Pharmacia) analysed by anti-LAG3 Western blot in order to assess their size. The apparent molecular weights were those expected.

The DNA of the Dα-LAG-3_{(D1D4)}Ig/E7 clone is that used for the stable transfection into CHO-dhfr⁻ cells.

### 1.3) Fusions Between hLAG-3Ig and HIV-1 gag-nef

This HIV-1 antigen was bound to hLAG-3Ig (D1D4 or D1D2), either at the C— or N-terminal end, and cloned into the expression vectors described above. This antigen and IgG1 without LAG-3 were also fused as a control (FIG. 4).

The HIV-1 antigen chosen in order to prepare this vaccinal recombinant protein is an optimised fusion product of gag p17, gag p24 and a part of nef.

### 1.3.1) Gag-nef Sequence Used

The sequence of the gag p17, gag p24 and nef chimeric protein was defined in order to have the greatest chance of being detected in European patients (B strains).

In order to do this, we compared for each protein the peptide sequences obtained at the website http://hiv-web.lanl.gov/content/hiv-db/CONSENSUS/MGROUP2002-Aug.html.

The alignment of the following sequences is shown in FIG. 3:
The ancestral sequence of B strain (the theoretical sequence from which the current viruses of B group are derived, was performed based on the phylogenetic tree).
The consensus sequence of B strain (consensus between all the current sequences of the B group, not taking into account their representation).
The sequence of the LAI strain (1^{st} European isolate)

The sequence chosen for each protein p24, p17 and nef corresponds to the ancestral sequence, except when an amino acid differs both from LAI and consensus, these two sequences being identical for this amino acid. We consider in this case that there is a better chance to find the consensus and LAI sequence in the current population than the ancestral sequence (FIG. 3). These three peptide sequences were subsequently placed end to end. The first 60 amino acids of nef were deleted, since they do not contain any major T epitope detected in patients and are not responsible for the cytopathogenic effect of nef.

The DNA sequence corresponding to the peptide sequence thus obtained for the gag-nef chimera was optimised for expression in hamster cells (CHO-K1 cells) by ATG-Biosynthetics Company. Restriction sites were added at the 5′ and 3′ ends of gagnef in order to allow sub-cloning. The gene was subsequently synthesised by ATG-Biosynthetics Company and supplied in the pCR4topo vector.

### 1.3.2) Cloning of hLAG-3Ig-gagnef and gagnef-hLAG-3Ig into the Expression Vectors

The cloning strategy is summarised in FIG. 4.

Cloning of hLAG-3Ig-gagnef into pCDNA3.1:

Gag-nef was sub-cloned from pCR4topo-gagnef into the pCDNA3.1-hLAG-3_{(D1D4)}Ig and pCDNA3.1-hLAG-3_{(D1D2)}Ig vectors between XhoI and XbaI.

The pCDNA-hLAG-3_{(D1D4)}Ig-gagnef and pCDNA-hLAG-3_{(D1D2)}Ig-gagnef expression vectors were verified by enzymatic digestion.

Cloning of gagnef-hLAG-3Ig into pSECtag-hybroB:

This cloning was performed in three steps:
First step: sub-cloning of gag-nef from pCR4topo-gagnef into pSECtag-hygroB between Hind3 and Not1. A pSEC-gagnef cloning intermediate was thus obtained.
Second step: cloning of hLAG-3Ig (D1D4 and D1D2) into pSECtaghygroB at XhoI. This step gives a cloning intermediate for obtaining large quantities of XhoI-digested insert. Indeed, if the PCR products had been directly digested by XhoI, a large number of undigested fragments would have be subsequently blunt cloned, resulting in false positives impossible to eliminate but by sequencing.

hLAG-3_{(D1D4)}Ig and hLAG-3_{(D1D2)}Ig were amplified by PCR from pCDNA3-LAG-3_{(D1D4)}-IgG1 and pCDNA3-LAG-3_{(D1D2)}-IgGI, respectively, with the following pair of oligonucleotides:

Oligo 5 (Lag3 5′-D1XhoI):

| | |
|---|---|
| CCGCTCGAGTCCAGCCAGGGGCTGAG | (SEQ ID No. 13) |

containing a XhoI site and the 5′ region of domain 1, without the signal peptide.

Oligo 17 (IgGI 3′-stopXhoI):

| | |
|---|---|
| CCGCTCGAGTCATTTACCCGGGGACAG | (SEQ ID No. 14) |

containing the 3′ region of IgGI with the stop codon and a XhoI site.

IgGI was amplified by PCR from pCDNA3-LAG-3_{(D1D4)}-IgG1 with the following pair of oligonucleotides:

Oligo 11 (IgG1 5′XhoI):

| | |
|---|---|
| CCGCTCGAGCGAGGGTGAGTACTAAGC | (SEQ ID No. 15) |

containing a XhoI site and the 5′ region of the A intron of IgGI.

Oligo 17 (IgG1 3′-stopXhoI):

| | |
|---|---|
| CCGCTCGAGTCATTTACCCGGGGACAG | (SEQ ID No. 16) |

containing the 3′ region of IgG1 with the stop codon and a XhoI site.

The PCR products were digested by XhoI before purification. They were subsequently ligated into pSECtag-hygroB previously digested by XhoI.
Third step: cloning of hLAG-3_{(D1D4)}Ig and hLAG-3_{(D1D2)}If into pSEC-gagnef.

The hLAG-3_{(D1D4)}Ig, hLAG-3_{(D1D2)}Ig and IgGI inserts were removed from pSECtag-hybroB by digestion with XhoI, the 5′ sticky ends were blunted with PNK. The pSEC-gagnef vector was digested with NotI and the cohesive ends were also blunted with PNK. Inserts and purified vectors were ligated.

The linker sequences between these expression vectors' inserts were sequenced in order to verify the integrity of the reading frame.

Cloning of gagnef into Dα-LAG3-D1D4-ΔEK-hIgG1 Fusion:

The destination vector is that used by Henogen for the production of LAG-3_{(D1D4)}Ig, Dα-LAG3-D1D4-ΔEK-hIgG1 Fusion.

The gagnef coding fragment is derived from pCDNA3-LAG-3_{(D1D4)}Ig-gagnef.

The cloning of gagnef into Dα-LAG3-D1D4-ΔEK-hIgG1 Fusion was performed in two steps:

In the first step, the LAG-3_{(D1D4)}-Ig/fragment was inserted into pCDNA-LAG-3_{(D1D4)}Ig-gagnef.

The Dα-LAG3-D1D4-ΔEK-hIgG1 vector was cleaved with XhoI and the cohesive ends were blunted with T4 polymerase. The fragment corresponding to the Dα vector without the XhoI blunt/XhoI blunt insert was retained as the final destination vector. The XhoI blunt/XhoI blunt insert was digested with BsrGI, cleaving the C intron of the coding sequence for Ig in order to eliminate the last 300 base pairs of the Ig coding sequence including the stop codon (LAG-3_{(D1D4)}Ig/XhoI blunt/BsrGI insert).

The pCDNA3-LAG-3_{(D1D4)}Ig-gagnef vector was digested with EcoRI (EORI site contained in the Multiple Cloning site of pCDNA3 upstream from the cloning site) and the cohesive ends were blunted with T4 polymerase. The plasmid thus linearised was cleaved with BsrGI in order to eliminate the sequence coding for LAG-3_{(D1D4)} and the 5′ sequence coding for Ig and retain the 300 last base pairs of the sequence coding for Ig (pCDNA EcoRI blunt/BsrGI Ig-gagnef). The LAG-3_{(D1D4)}Ig/XhoI blunt/BsrGI fragment was ligated into pcDNA3 EcoRI blunt/BsrGI Ig-gagnef.

Following restriction analysis of the obtained clones, a clone containing pCDNA3-LAG-3_{(D1D4)}Ig/gagnef was selected.

In the second step, LAG-3_{(D1D4)}Ig/gagnef was cloned into Dα.

The entire LAG-3_{(D1D4)}Ig/gagnef insert contained in pCDNA3 was removed by digestion with PmeI (2 sites surrounding the Multiple cloning site of pCDNA3, blunt end) and cloned into Dα without the XhoIblunt/XhoIblunt insert previously prepared by cleaving and dephosphorylated.

The clones comprising the LAG-3_{(D1D4)}Ig/gagnef insert in Dα in the proper direction were selected by restriction analysis.

The DNA of one of the Dα-LAG-3_{(D1D4)}Ig/gagnef clones was transformed back into the DH5α strain and prepared using maxiprep Endofree (Qiagen) and used for the transient transfection of CHO-K1 cells in order to check the translation product of the plasmid. The original Dα-LAG3-D1D4-ΔEK-hIgG1 Fusion and pCDNA-LAG-3_{(D1D4)}Ig vectors were used as positive controls and the Dα-vector, the insert of which had been eliminated using XhoI followed by religation, was used as negative control. Twenty-four hours post-transfection, the supernatants were assayed for LAG-3 using specific ELISA. In parallel, the recombinant proteins present in the supernatants and cell lysates were precipitated by A-sepharose protein (Pharmacia) and analysed with anti-LAG3 western blot in order to assess their size. The apparent molecular weights were such as expected.

The DNA of the selected Dα-LAG-3_{(D1D4)}Ig/gagnef clone is that used for the stable transfection of CHO-dhfr⁻ cells.

### 2) ESTABLISHMENT OF STABLE CHO CELL LINES EXPRESSING THE FUSION PROTEINS

### 2.1) Establishment of Stable Lines Expressing the Fusion Proteins from pCDNA3 and pSEC Vectors.

The expression vectors previously constructed in pCDNA3 or pSEC were transfected into CHO-K1 cells in order to obtain stable producing lines expressing the recombinant proteins of interest.

### 2.1.1) Transfection Method and Isolation of the Clones

### 2.1.1.1) Plasmid Digestion

In order to maximise the chances of obtaining stable transfectants expressing the protein of interest, the expression vectors were linearised before transfection. The restriction enzyme chosen was Bg12, which digests the pCDNA and pSEC vectors at position 1, but none of the inserts hLAG-3Ig, gagnef or E7. Digestion was performed with 10 μg of vector and 30 U of enzyme and the DNA was subsequently precipitated and taken up in 20 μL of H₂O UP.

However, a comparative study showed that this linearization step is not crucial since the number of positive stable transfectants obtained differs little whether the was linearised or not.

### 2.1.1.2) Transfection

Transfections were all performed between 3 and 4 times independently in CHO-K1 cells (ECCAC Ref. No. 85081005) having a number of runs from 8 to 14.

Briefly, semi-confluent CHO-K1 cells were transfected in 6-well plates with 2 μg of plasmid, using 5 μL of Lipofectamine (GIBCO).

### 2.1.1.3) Selection of Resistant Clones

24 hours after transfection, the cells were lysed with trypsine and inoculated into a 150 mm round dish, in the presence of medium containing the selection antibiotic. The selection antibiotic is G418 at 0.5 mg/mL for the pCDNA vectors and hygromycine B at 0.4 mg/mL for the pSEC vectors. The medium was changed every 2-3 days until appearance of isolated cell clones (1 to 2 weeks).

The cell clones which were resistant to the antibiotic, having therefore integrated the plasmid, were taken up manually under the microscope with a P200 pipette and transferred to 96-well plates. Each clone was subsequently tested for the expression of the protein of interest and the positive clones were subsequently amplified.

### 2.1.1.4) Designation of the Clones

The clones were named according to a code allowing identification of the date of transfection from which they resulted and the plasmid that they contain.

The first two figures correspond to the day and month of transfection, followed by a letter corresponding to the plasmid transfected, followed by the number of the clone. For example, a clone obtained from the transfection of 7^{th} January, with the pCDNA plasmid will be named 07_{—}01_Ax (x being the clone number).

A letter was assigned to each plasmid according to the following nomenclature:

A: pCDNA

B: pCDNA-hLAG-3_{(D1D4)}Ig

C: pCDNA-hLAG-3_{(D1D2)}Ig

D: pCDNA-hLAG-3_{(D1D4)}Ig-E⁷

E: pCDNA-hLAG-3_{(D1D2)}Ig-E⁷

F: pSEC

G: pSEC-E7-hLAG-3_{(D1D4)}Ig

H: pSEC-E7-hLAG-3_{(D1D2)}Ig

I: pSEC-E7-IgG1

J: pCDNA-hLAG-3_{(D1D4)}Ig-gagnef

K: pCDNA-hLAG-3_{(D1D2)}Ig-gagnef

L: pSEC-gagnef-hLAG-3_{(D1D4)}Ig

M: pSEC-gagnef-hLAG-3_{(D1D2)}Ig

N: pSEC-gagnef-IgG1

### 2.1.2) Selection of the Highest hLAG-3Ig-E7 Recombinant Protein-Producing Clones

The first series of plasmids transfected were pCDNA-LAG-3_{(D1D4)}Ig-E⁷, pCDNA-LAG-3_{(D1D2)}Ig-E7, in addition to pCDNA-LAG-3_{(D1D4)}Ig and pCDNA-LAG-3_{(D1D2)}Ig, as positive controls without E7.

### 2.1.2.1) Analysis of the Transient Transfection Efficacy

The transfection efficacy of the cells was initially assessed by FACS following intracellular marking with an anti-LAG3 antibody (17B4), 24 hours after transfection. The transient transfection efficacy was showed to be good for the plasmids pCDNA-hLAG-3_{(D1D4)}Ig, pCDNA-hLAG-3_{(D1D2)}Ig and pCDNA-hLAG-3_{(D1D2)}Ig-E7 (between 40 and 50% of positive cells) but much less for pCDNA-hLAG-3_{(D1D4)}Ig-E7.

### 2.1.2.2) Testing of Stably Transfected Clones

This series of transfections was repeated 4 times: on Jan. 1, 2003, Jan. 14, 2003, Jan. 21, 2003 and Mar. 27, 2003.

The supernatants of each transfected cell clone were tested by ELISA with anti-LAG-3, undiluted or diluted twice as appropriate.

The best clones of each transfection series were amplified and two ampoules per clone were frozen. The supernatants of these clones were subsequently compared by ELISA in order to keep only the highest producing clones.

Clones frozen and subsequently compared with one another:

For pCDNA: 7-1-A1, 14-1-A2, 21-1-A2

For pCDNA-hLAG-3_{(D1D4)}Ig: 7-1-B1, 7-1-B3, 7-1-B4, 14-1-B8, 14-1-B14, 21-1-B11, 27-6-B6

For pCDNA-hLAG-3_{(D1D2)}Ig: 7-1-C1, 7-1-C2, 14-1-C12, 14-1-C16, 21-1-C8, 27-5-C5

For pCDNA-hLAG-3_{(D1D4)}Ig-E7: 7-1-D3, 14-1-D4, 14-1-D8, 21-1-D1, 27-5-D3

For pCDNA-hLAG-3_{(D1D2)}Ig-E7: 7-1-E1, 7-1-E3, 7-1-E5, 14-1-E9, 14-1-E11, 21-1-E6, 27-5-E8.

The best clones were also compared by Western blot analysis of their supernatants. The combined testing allowed the selection for each transfected plasmid of the clone which would be amplified and serve as producing cell for the recombinant proteins.

The clones selected as recombinant protein-producing lines were:

For pCDNA: 7-1-A1

For pCDNA-hLAG-3_{(D1D4)}Ig: 7-1-B4

For pCDNA-hLAG-3_{(D1D2)}Ig: 21-1-C8

For pCDNA-hLAG-3_{(D1D4)}Ig-E7: 7-1-D3

For pCDNA-hLAG-3_{(D1D2)}Ig-E7: 14-1-E11

As expected, hLAG-3_{(D1D4)}Ig-E7 migrates slightly higher than hLAG-3_{(D1D4)}Ig (FIG. 5). The two bands observed correspond to the monomeric and dimeric forms of hLAG-31g, the reduction by heating in the presence of β-mercaptoethanol not having been sufficiently effective.

### 2.1.2.3) Freezing of the Producing Cell Banks

One of both stock ampoules having been frozen following a limited number of runs was thawed and reamplified until confluence was reached in a 175 cm² flask, from which 5 ampoules were frozen as “Master Cell Bank′ on Mar. 3, 2003. The number of runs since isolation of the clones is then between 5 and 7 according to line.

Five other ampoules were subsequently frozen from a 175 cm² flask as “Working Cell Bank” on May 3, 2003.

### 2.1.3) Selection of the Highest E7-hLAG-31g Recombinant Protein-Producing Clones

### 2.1.3.1) Analysis of the Transient Transfection Efficacy

The transfection efficacy of the cells was assessed by FACS following intracellular marking with an anti-human IgG antibody coupled to FITC, 24 hours after transfection. The efficacy of transient transfection is higher for pSEC-E7-hLAG-3_{(D1D4)}Ig than for pSEC-E7-hLAG-3_{(D1D2)}Ig or pSEC-E7-IgG1. Although the quality of preparation of the DNA is better (the SIGMA genelute kit was replaced by the midiprep Qiagen kit), the efficacy of transient transfection of the pSEC plasmids is less than that obtained with the pCDNA plasmids (less than 10% versus 50%).

### 2.1.3.2) Testing of Stably Transfected Clones

This series of transfections was repeated four times: on Apr. 23, 2003, Jul. 5, 2003, Jun. 19, 2003 and Jun. 27, 2003.

The supernatants of all the cell clones transfected with pSEC-E7-hLAG-3_{(D1D4)}Ig and pSEC-E7-hLAG-3_{(D1D2)}Ig were tested undiluted by ELISA with anti-LAG-3. The OD of the supernatants of cells expressing E7-LAG3 were much lower than those of cells expressing LAG3-E7.

The cell clones transfected with pSEC-E7-IgG1 expressing a fusion product not containing LAG3 can obviously not be analysed by ELISA. These were all tested by FACS after intracellular marking with a human anti-IgG antibody coupled to FITC.

The best clones of each series of transfections were amplified and two ampoules per clone were frozen. The following frozen clones were compared with one another:

For pSEC: 23-04-F4, 07-05-F1

For pSEC-E7-hLAG-3_{(D1D4)}Ig: 23-04-G3, 07-05-G27, 07-05-G32, 19-06-G8, 19-06-G40

For pSEC-E7-hLAG-3_{(D1D2)}Ig: 23-04-H10, 07-05-H11

For pSEC-E7-IgG1: 23-04-I12, 07-05-I13, 19-06-I4, 19-06I19.

The supernatants of these clones were subsequently compared with each other by ELISA or by intracellular marking, in order to keep only the highest producing lines. The clones chosen as recombinant protein-producing lines were:

For pSEC: 07-05-F1;

For pSEC-E7-hLAG-3_{(D1D4)}Ig: 19-06-G8

For pSEC-E7-hLAG-3_{(D1D2)}Ig: no clone was amplified since the OD of the supernatants was low as compared to the E7-D1D4 clones. Both ampoules of 07-05-H11 were nevertheless kept in liquid nitrogen;

For pSEC-E7-IgG1: 07-05-I13.

### 2.1.3.3) Freezing of the Producing Cell Banks

Five ampoules of the 19-06-G8 and 07-05-I13 lines were frozen from a 175 cm² flask at confluence as “Master Cell Bank” on May 17, 2003. The number of runs since the isolation of the clones was between 5 and 8 in this case, according to the line considered.

A further five ampoules were subsequently frozen from a 175 cm² flask as a “Working Cell Bank” on Jul. 21, 2003.

2.1.4) Selection of the Highest hLAG-3Ig-gagnef Recombinant Protein-Producing Clones

### 2.1.4.1) Analysis of the Transient Transfection Efficacy

The transfection efficacy was assessed by FACS following intracellular marking with an anti-human IgG antibody coupled to FITC on transiently transfected cells. The transient transfection efficacy is approximately 10%.

### 2.1.4.2) Testing of the Stably Transfected Clones

This series of transfections was repeated 3 times: on May 7, 2003, Jun. 19, 2003 and Jun. 27, 2003.

The supernatants of each transfected cell clone were tested undiluted by ELISA with anti-LAG-3.

The best clones of each transfection series were amplified; two ampoules per clone were frozen. The following frozen clones were subsequently compared with one another:

For pCDNA-hLAG-3_{(D1D4)}Ig-gagnef: 07-05-J2, 07-05-J23, 07-05-J53, 07-05-J71, 19-06-J18, 19-06-J32, 19-06-J48

For pCDNA-hLAG-3_{(D1D2)}Ig-gagnef: 07-05-K19, 07-05-K71, 19-06-K33, 19-06-K43, 19-06-K44, 17-06-K7

These clones were subsequently compared with one another by ELISA of their supernatants or by intracellular marking, in order to keep only the highest producing lines. The clones selected as recombinant protein-producing lines were:

For pCDNA-hLAG-3_{(D1D4)}Ig-gagnef: 07-05-J53

For pCDNA-hLAG-3_{(D1D2)}Ig-gagnef: 07-05-K19 and 27-06-K7.

### 2.1.4.3) Freezing of the Producing Cell Banks

Five ampoules of the 07-05-J53 and 07-05-K19 lines from a 175 cm² flask were frozen, in addition to 3 ampoules of 27-06-K7 from a 185 cm² flask as “Master Cell Bank” on May 17, 2003. The number of runs since isolation of the clones was between 4 and 8 according to the line considered.

Five or three additional ampoules were likewise frozen as “Working Cell Bank” on Jul. 21, 2003.

### 2.2) Establishment of Stable Lines Expressing the Fusion Proteins from Dα Vector.

The Dα-LAG-3_{(D1D4)}Ig/E7 and Dα-LAG-3_{(D1D4)}Ig/gagnef expression vectors constructed were transfected into CHO-dhfr⁻ cells in order to obtain sable producing lines expressing the recombinant proteins of interest.

### 2.2.1) Transfection Method and Isolation of the Clones

### 2.2.1.1) Transfection

CHO-dhfr⁻ cells (DSMZ ACC126), used for the transfection of the constructs in Dα vector, were incubated in the presence of ribonucleosides and deoxyribonucleosides (medium MEMα RN/RdN+, GIBCO 22571-020). The CHO-dhfr⁻ cells were inoculated on the previous day into 25 cm² flasks and transfected on 25 Jul. 2004 with 1 μg Dα-LAG-3_{(D1D4)}Ig/E7 or Dα-LAG-3_{(D1D4)}Ig/gagnef plasmid, using 2.5 μL of lipofectamine 2000 (In-vitrogen). Transfections of the original Dα-LAG-3-D1D4-ΔEK-hIgG1 Fusion and PEGFP (Clontech) vectors were used as positive and negative controls, respectively. The transfection medium was replaced 6 hours after transfection with MEMα RN/RdN+ medium.

### 2.2.1.2) Selection of Resistant Cells

Two days after transfection, the cells were lysed with trypsine and inoculated for each transfection into 4 96-well plates at 5000 cells/well in MEMα RN/RdN-selection medium (GIBCO 22561-021). After approximately one week, the supernatant of each well, each representing a pool of cells, was assayed by ELISA with anti-LAG-3. The most productive pools were amplified for freezing and kept in culture in the presence of Methotrexate in order to allow amplification of the plasmid-derived sequences (and thus of the genes coding for the protein of interest).

All the steps for selecting and expanding the pools and clones were conducted in a medium containing endotoxin-free foetal calf serum (GIBC016000-044) and were dialysed in order to avoid the contamination of nucleosides from the serum.

### 2.2.2) Selection of the Highest hLAG-3Ig/E7 Recombinant Protein-Producing Clones

### 2.2.2.1) Selection of the Pools of the Most Productive hLAG-3Ig/E7 Transfectants

Among the 400 pools tested by ELISA with anti-LAG-3, 6 were far higher producers than the others and were selected (pools Nos. 3, 4, 9, 11, 20, 21). Two ampoules of each of these pools were frozen.

### 2.2.2.2) Gene Amplification by Methotrexate

These pools were inoculated in 6-well plates at 150.000 cells/well and cultivated with 50 nM methotrexate. The quantity of hLAG-3Ig/E7 in the supernatants of pools 9 and 11 was increased in the presence of methotrexate. The doses of methotrexate were therefore increased to 150 and 250 nM.

The pool 9 was resistant to 250 nM methotrexate and the quantity of hLAG-3Ig/E7 produced was higher. A limiting dilution experiment was therefore performed under these conditions on 11 Sep. 0204. Two clones derived from this pool were selected (Nos. 9-23 and 9-26) and frozen on 8 Oct. 2004.

The pool 11 was resistant to 250 nM methotrexate and the quantity of hLAG-3Ig/E7 produced was greater. A limiting dilution experiment was therefore performed under these conditions on 3 Sep. 2004. Five clones derived from this pool were selected (Nos. 11-1, 11-2, 11-5, 11-6, 11-10) and frozen on 6 Oct. 2004.

The adaptation of these clones in chemically defined media (containing a low level of exogenous protein) and supplemented with 2 mM butyrate (a differentiating agent known to increase production of the recombinant proteins) was assessed in terms of hLAG-3Ig/E7 protein produced. Based on its production level in serum-free media, the quality of the protein produced (Western blot and development with an anti-LAG-3 antibody) and its growing capacity, the clone hLAG-3Ig/E7 #11-5 was selected. Twenty ampoules of this clone were frozen on 5 Nov. 2004.

### 2.2.3) Selection of the Highest hLAG-3Ig/gagnef Recombinant Protein-Producing Clones

### 2.2.3.1) Selection of the Most Productive Pools of hLAG-3Ig/gagnef Transfectants

Among the 400 pools tested by ELISA with anti-LAG-3, 24 were selected (pools numbered 1 to 24) and frozen.

### 2.2.3.2) Gene Amplification with Methotrexate

These pools were inoculated in 6-well plates at 150000 cells/well and cultivated with 50 nM methotrexate. Eleven pools resistant to 50 nM methotrexate were cultivated with 150 and 250 nM methotrexate.

The pool 1 was resistant to 250 nM methotrexate and the quantity of hLAG-3Ig/gagnef produced was higher. A limiting dilution test was therefore performed under these conditions on 6 Sep. 2004. Five clones derived from this pool were selected (Nos. 1-14, 1-21, 1-49, 1-56, 1-100) and frozen on 6 Oct. 2004.

The pool 6 was resistant to 250 nM methotrexate and the quantity of hLAG-3Ig.gagnef produced was higher. A limiting dilution test was therefore performed under these conditions on 11 Sep. 2004. Five clones were selected (Nos. 6-15, 6-55, 6-57, 6-58, 6-65) and frozen on 5 Oct. 2004.

Adaptation of these clones in chemically defined media supplemented with 2 mM butyrate was assessed in terms of hLAG-3Ig/gagnef protein produced. According to productivity in serum-free media, quality of the protein produced (Western blot and developing with an anti-LAG-3 antibody) and growing capacity, the clone hLAG-3Ig/gagnef No. 1-21 was selected. Twenty ampoules of this clone were frozen on 29 Nov. 2004.

### 3) PRODUCTION AND PURIFICATION OF THE FUSION PROTEINS

The hLAG-3Ig-E7, hLAG-3Ig/E7 and hLAG-3Ig/gagnef proteins and hLAG-3Ig as control were produced and purified.

### 3.1) Production of the Fusion Proteins

### 3.1.1) Production of Large Volumes of Supernatant from hLAG-3Ig-E7 and hLAG-3Ig Producing Lines

The cell lines producing hLAG-3_{(D1D4)}Ig (CHO 7-1-BA), hLAG-3_{(D1D4)}Ig-E⁷ (CHO 7-1-D3), hLAG-3_{(D1D2)}Ig (CHO 21-1-C8) and hLAG-3_{(D1D2)}Ig-E7 (CHO 14-1-E11) proteins were cultivated on a larger scale in order to obtain volumes of supernatant containing a few milligrams of protein (between 1.3 and 1.6 liters of supernatant per protein).

All cultures were performed with adherent cells incubated in 175 cm² flasks with 80 mL of medium per flask. The medium used was Ham F12 (Invitrogen), added with 10% FCS (batch S135) without any selective antibiotic.

The cells were diluted either at 1/10^{th} and cultivated for 4 days, or at ⅓^{rd} and cultivated for 2 days. Harvesting of the supernatant was therefore performed before the medium became too yellow and the cells detached.

The recombinant protein concentrations in the production batches were:
hLAG-3_{(D1D4)}Ig (CHO 7-1-BA): 1.4 mg/L
hLAG-3_{(D1D4)}Ig-E7 (CHO 7-1-D3): 0.33 mg/L
hLAG-3_{(D1D2)}Ig (CHO 21-1-C8): 4 μg/L
hLAG-3_{(D1D2)}Ig-E7 (CHO 14-1-E11): 5 μg/L

Production of the recombinant proteins containing only 2 Ig domains of hLAG-3 was therefore much more effective than with 4 Ig domains of hLAG-3.

### 3.1.2) Production of Large Volumes of Supernatant from hLAG-3Ig/E7 No. 11-5 and hLAG-3Ig/gagnef No. 1-21 Clones

The increase in the biomass of the hLAG-3Ig/E7 No. 11-5 and hLAG-3Ig/gagnef No. 1-21 clones was obtained with adherent cells in the presence of serum. At confluence, the cells were washed with PBS and cultivated in ProCHO4-CDM media (Cambrex BE12-029Q) supplemented with 250 nM methotrexate and 2 mM butyrate at 30° C. The medium was recovered every 24 or 36 hours. Several productions were thus obtained and the quantities produced in the supernatants were generally greater than 2 mg/L for the hLAG-3Ig-E7 and 1 mg for the hLAG-3Ig-gagnef fusion proteins in this expression system.

### 3.2) Purification of hLAG-3Ig-E7 and hLAG-3Ig, hLAG-3Ig/E7 and hLAG-3Ig/gagnef

The hLAG-3_{(D1D4)}Ig, hLAG-3_{(D1D4)}Ig-E⁷, hLAG-3_{(D}₁D₂)Ig, hLAG-3_{(D1D2)}Ig-E⁷, hLAG-3Ig/E7 and hLAG-3Ig/gagnef recombinant proteins were purified on a column of protein A from the batches of supernatants produced.

### 3.2.1) Purification Protocol Used

Purification of the recombinant proteins from the culture supernatant was performed on a column of protein A (Pharmacia ref-17-5079-01) balanced with PBS.

The culture supernatant was filtered on 0.22 μm filter. It was loaded on the column of protein A, previously balanced with PBS, using a FPLC system from Pharmacia. During purification of hLAG-3_{(D1D4)}Ig, hLAG-3_{(D1D4)}Ig-E7, hLAG-3_{(D1D2)}Ig, hLAG-3_{(D1D2)}Ig-E7, the proteins unbound to protein A were washed with 10 ml of PBS and the recombinant protein was then eluted using a gradient of 0.1 M glycerine buffer (pH between 4 and 2.7). 1 ml fractions were collected.

The UV detector indicated the presence of an elution peak when the gradient reaches approximately 20% of pH 2.7 buffer. The elution profile was the same for the four recombinant proteins.

The fractions containing the purified protein were pooled and subsequently desalted in a PBS buffer on a desalting column (Hi-trap desalting 5 ml from Pharmacia). The proteins thus obtained were concentrated (before their use in the functional tests) on a concentrator (Vivascience ref. V50201 cut-off 10 kDa) and sterilised by filtration over a SpinX column (ref. Costar 8160). The product was aliquoted and frozen at −80° C.

For the hLAG-3Ig/E7 and hLAG-3Ig/gagnef recombinant proteins, after washing in PBS and 0.1 M glycine buffer, pH 4, the protein was directly eluted in 0.1 M glycine buffer, pH 2.7 and dialysed against PBS, filtered at 0.2 μm, aliquoted and stored at −80° C.

### 3.2.2) Yields of the Purification Steps

The product of each purification, desalting and Centricon concentration step was analysed by ELISA with anti-hLAG-3Ig.

Table 1 summarises the concentrations obtained by ELISA and yields of each purification step of the hLAG-3_{(D1D4)}Ig, hLAG-3_{(D1D4)}Ig-E⁷, hLAG-3_{(D1D2)}Ig and hLAG-3_{(D1D2)}Ig-E⁷ recombinant proteins.

TABLEAU 1

| | | | | | Fractions | Fractions | Fractions | |
|---|---|---|---|---|---|---|---|---|
| | | Supernatant | Eluent | Washing | 7-8-9 | 10-11-12 | 13-14 | Yield |
| hLAG- | Concentration | 1.45 | 0.02 | 0 | 352 | 191 | 45.2 | 78.3% |
| 3_{(D1D4)}Ig | (μg/mL) | | | | | | | |
| | Total quantity | 2169 | 31.8 | 0 | 1698.6 | | | |
| | (μg) | | | | | | | |
| | After desalting | | | | 170 μg/mL (for 12 ml) | | | 10% |
| | After | | | | 111 μg/mL (for 12 ml) | | | |
| | concentration | | | | | | | |
| hLAG- | | 0.33 | 0.02 | 0 | 45.2 | 67.2 | 35.2 | 75.8% |
| 3_{(D1D4)}Ig- | Total quantity | 514.6 | 34.8 | 0 | 390 | | | |
| E7 | (μg) | | | | | | | |
| | After desalting | | | | 39 μg/mL (for 12 ml) | | | 25% |
| | After | | | | 84 μg/mL (for 12 ml) | | | |
| | concentration | | | | | | | |
| hLAG- | Concentration | 0.004 | 0.003 | 0 | <0.25 | <0.25 | <0.25 | / |
| 3_{(D1D2)}Ig | (μg/mL) | | | | | | | |
| E7-hLAG- | Concentration | 0.006 | 0.006 | 0 | <0.25 | <0.25 | <0.25 | / |
| 3_{(D1D2)}Ig- | (μg/mL) | | | | | | | |
| E7 | | | | | | | | |

The yield obtained during purification of hLAG-3_{(D1D4)}Ig and hLAG-3_{(D1D4)}Ig-E7 on a protein A column is about 77%, which is relatively satisfactory.

Dilution of the product by a factor 1.5 during the replacement of the buffer on the desalting column is normal.

Table 2 summarises the quantities obtained by BCA protein assay (Perbio), in addition to the quantity of endotoxins estimated by LAL (Cambrex) in the purified hLAG-3Ig/E7 and hLAG-3Ig/gagnef proteins.

TABLE 2

| | | Quantity of | Endotoxin |
|---|---|---|---|
| | Purification | purified protein | level |
| | date | (mg) | (EU/mg) |
| hLAG-3Ig/E7 | 16^{th} November 04 | 1.8 | 1.3 |
| E7 | 1^{st} December 04 | 2.8 | 1.5 |
| | 11^{th} January 05 | 1.08 | 0.5 |
| | 28^{th} January 05 | 2.40 | 1.4 |
| hLAG-3Ig/ | 10^{th} December 04 | 1.25 | 0.3 |
| gagnef | 10^{th} January 05 | 2.11 | 0.43 |
| | 21^{st} January 05 | 1.60 | 0.65 |

The use of serum-free medium allowed the level of endotoxins to be reduced well below the limits imposed for use of our invention on dendritic cells in vitro and in animals.

### 3.2.3) Analysis of the Purified Products by SDS-PAGE

The nature and purity of the purified products of the four hLAG-3_{(D1D4)}Ig, hLAG-3_{(D1D4)}Ig-E7, hLAG-3_{(D1D2)}Ig and hLAG-3_{(D1D2)}Ig-E⁷ recombinant proteins were analysed by SDS-PAGE. The gels containing 10% acrylamide were either stained with Coomassie blue or transferred to nitrocellulose for analysis by Western blot with anti-hLAG3 and anti-E7 (examples on FIG. 5).

The hLAG-3_{(D1D4)}Ig protein purified on a protein A column migrated in the same manner as the purified hLAG-3_{(D1D4)}Ig protein (FIG. 5). On the other hand, two proteins of smaller size were also present in large quantities in our purification product. They correspond to the bovine immunoglobulins present in the serum used for the cell culture.

hLAG-3_{(D1D4)}Ig bound to E7 migrated slightly less rapidly, which was expected. In order to confirm the presence of the E7 fragment, Western blot analysis with an anti-E7 antibody was performed. The hLAG-3_{(D1D4)}Ig-E7 fusion was shown.

The nature and purity of the purification products of each batch of hLAG-3_{(D1D4)}Ig/E7, hLAG-3_{(D1D4)}Ig/gagnef were analysed by staining with Coomassie blue and anti-LAG-3 Western blot (example on FIG. 8).

As expected, the purified hLAG-3_{(D1D4)}Ig/E7 and hLAG-3_{(D1D4)}Ig/gagnef proteins had an apparently higher molecular weight than hLAG-3_{(D1D4)}Ig. The gagnef fragment being larger than the E7 fragment, the hLAG-3_{(D1D4)}Ig/gagnef fusion protein had the slowest migration (FIG. 8). The use of serum-free medium allowed the contaminating proteins to be eliminated.

### 4) FUNCTIONALITY TEST OF THE FUSION PROTEINS

The hLAG-3_{(D1D4)}Ig, hLAG-3_{(D1D4)}Ig-E7, hLAG-3_{(D1D4)}Ig and hLAG-3_{(D1D4)}Ig-E⁷ proteins purified in this manner were tested for their ability to bind the class II MHC, for their capacity to induce maturation of the dendritic cells, to be internalised by the dendritic cells and for their capacity to induce a specific CD4 and/or CD8 T-cell response.

### 4.1) Binding to the Class II MHC

The ability of the recombinant proteins to bind the class II MHC was assessed by measuring the binding of the proteins to LAZ-509 cells (human B cells transformed by EBV, strongly expressing the class II MHC). Binding was revealed by means of an human anti-Ig antibody coupled to FITC. The intensity of the FITC marking of the LAZ-509 cells, proportional to the binding of the recombinant protein to the class II MHC, was quantified by FACS (FIGS. 6 and 9).

The hLAG-3_{(D1D4)}Ig-E7 fusion protein binds to class II MHC in a similar manner as hLAG-3_{(D1D4)}Ig produced and purified under the same conditions (FIG. 6).

Likewise, the binding capacity of the hLAG-3_{(D1D4)}Ig/E7 and hLAG-3_{(D1D4)}Ig/gagnef proteins is similar to that of hLAG-3Ig (FIG. 9).

### 4.2) Maturation of Human Dendritic Cells by hLAG-3_{(D1D4)}Ig/E7 and hLAG-3_{(D1D4)}Ig/gagnef

Immature dendritic cells differentiated from peripheral blood monocytes (PBMC) were incubated for 2 days with human IgG1 cells (as a negative stimulation control) or hLAG-3_{(D1D4)}Ig/E7, hLAG-3_{(D1D4)}Ig/gagnef or hLAG-3Ig (10 μg/mL) proteins. Soluble CD40 ligand (sCD40L, 3 μg/mL), known to induce maturation of the dendritic cells, was used as the positive stimulation control. Activation of the dendritic cells was subsequently assessed by membrane expression of the CD40, CD80, CD83, and CD86 activation markers (example in FIG. 10). The LAG-3/E7 or LAG-3/gagnef fusion proteins induced maturation of the dendritic cells as do the positive controls, LAG-3Ig and sCD40L.

### 4.3) Internalisation of hLAG-3Ig-E7 in Human Dendritic Cells

Internalisation of the hLAG-3_{(D1D4)}Ig-E7 fusion protein in dendritic cells was tested.

Immature human dendritic cells purified from PBMC were incubated at 4° C. with 30 μg/mL recombinant proteins. The cells are subsequently placed at 37° C. for 15 minutes. It is known that under these conditions, hLAG3-Ig is internalised in the dendritic cells and induces their maturation.

Analysis by confocal microscopy showed that the hLAG-3_{(D1D4)}Ig-E7 protein was effectively internalised in immature human dendritic cells (FIG. 10). After having placed the slides at 37° C. for 15 minutes, the cells internalised the LAG-3Ig-E7 protein with specific marking in the dendritic cells, which is not found at 4° C. (negative internalisation control).

### 4.4) CD4 and CD8 T-cell Responses to the E7 and gag-nef Antigens

PBMC cells freshly collected from healthy donors were purified using standard Ficoll-Paque (Amersham Pharmacia Biotech AB).

The dendritic cells were prepared by cultivating the PBMC with 500 U/mL of GM-CSF (R&D Systems Inc. MN) and 500 U/mL of IL-4 (R&D Systems Inc. MN) for 7 days.

The dendritic cells were subsequently matured for 2 days in the presence of 2 μg/mL of anti-CD40 antibodies and 100 ng/mL of Poly IC (Sigma Aldrich).

The purified CD4 and CD8 T-cells were stimulated each week with dendritic cells for 2 hours with the hLAG-3_{(D1D4)}Ig/E7, hLAG-3_{(D1D4)}Ig/gagnef, E7, gagnef or hLAG-3Ig (10 μg/mL) proteins and irradiated at 35 Gy at a T-cell/dendritic cell ratio of 10/1 in the culture medium (CM: RPMI 1640 with 10% human AB serum, 10 nM L-glutamine and gentamycine). 1.10³ U/mL of IL-6 and 5 U/mL of IL-12 (R&D Systems Inc. MN) were added during the first week of culture. 20 U/mL of IL-2 (R&D Systems Inc. MN) and 10 ng/mL of IL-7 (R&D Systems Inc. MN) were added during the following two weeks.

The Elispot assays were used in order to quantify the effector cells secreting γ-interferon in response to the E7 or gag-nef antigens.

96-well plates with cellulose ester membrane (MultiScreen MAHA S4510; Millipore) were coated overnight with anti γ-interferon antibodies (MAB 285). The wells were washed and added with culture medium at 10% of human AB serum and the cells were added at four different concentrations. The proteins were added to each well and the plates were incubated overnight. On the following day, the medium was discarded and the wells were washed by adding a secondary biotinylated antibody (BAF285-Biotin). The plates were incubated for 2 hours and washed, and streptavidine-enzyme complex (Streptavidine-AP; Boehringer Mannheim GmbH) was added to each well. The plates were incubated at room temperature for 1 hour and the BCIP-NBT substrate of the enzyme (S3771: Promega France) was added to each well in the alkaline phosphatase buffer, pH 9.5 (100 mM Tris-HCl, 100 mM NaCl, 5 mM MgCl₂) and the plates were incubated at room temperature for 10 to 20 minutes. The reaction was terminated by washing with water as soon as dark violet spots appeared. The spots were counted using an automated image analyser ELISPOT Reader (AID Strasbourg, Germany).

The frequency of the CD4 or CD8 T effector cells secreting γ-interferon in response to the antigens may be calculated based on the number of cells forming spots and the frequency of the CD8 or CD4 T-cells in a population of lymphocytes by immunolabelling with an anti-CD8 or anti-CD4 antibody.

1) A coupling product, characterised in that it is comprised of a first class of protein of the antigen type and a second class of protein of the class II MHC ligand type, both classes of protein being coupled by one or several bonds which are stable in biological environments.

2) A coupling product according to claim 1, characterised in that both classes of protein are linked by covalent bonds.

3) A coupling product according to claim 2, characterised in that both classes of proteins are indirectly linked by covalent bonds via a linker or a linking molecule.

4) A coupling product according to claim 2, characterised in that both classes of protein form a fusion protein.

5) A coupling product according to claim 2, characterised in that the second class of protein is selected from the group comprising hLAG-3, its homologues, fragments and derivatives, and the mixtures thereof.

6) A coupling product according to claim 5, characterised in that the LAG-3 fragment is a soluble fragment.

7) A coupling product according to claim 6, characterised in that the LAG-3 fragment is selected from the group comprising D1-D2 and D1-D4 fragments.

8) A coupling product according to claim 1, characterised in that the first class of protein of the antigen type is selected from the group including antigens specific of a disease, the treatment of which requires a T lymphocyte response.

9) A coupling product according to claim 1, characterised in that the first class of protein of the antigen type is selected from the group comprising viral antigens, bacterial antigens, tumour antigens, parasitic antigens and mixtures thereof.

10) A coupling product according to claim 9, characterised in that the first class of protein of the antigen type is a viral antigen selected from the group comprising the HPV, HBV, HCV, HIV, EBV, CMV viruses and mixtures thereof.

11) A coupling product according to claim 10, characterised in that the first class of protein of the antigen type is selected from the group comprising the antigen E7 of HPV and the gag-nef antigen of HIV.

12) A coupling product according to claim 9, characterised in that the first class of protein of the antigen type is a bacterial antigen selected from the group of intracellular bacteria of tuberculosis, leprosy and listeria.

13) A coupling product according to claim 9, characterised in that the first class of protein of the antigen type is a tumour antigen selected from the group comprising CEA, Melan A, PSA, MAGE-3, HER2/neu, E6 and E7 proteins of HPV.

14) A vaccine composition characterised in that it comprises at least one coupling product according to claim 1, optionally combined with a pharmaceutically acceptable vehicle.

15) Use of a coupling product according to claim 1 for the preparation of a drug intended for treating infectious diseases and/or cancer.

16) Use according to claim 15, in which the treatment of infectious diseases and/or cancer implies an immune response via CD8+ T cells.

17) Use according to claim 16, in which the second class of protein of the MHC II ligand type is capable of inducing a antigen-specific immune response via the T cells.

18) Use of a coupling product according to claim 1, for the manufacture of a drug intended for the immunotherapy of cancer and the immunotherapy of infectious diseases.

19) Use of a coupling product according to claim 1 for the preparation of an immunogenic composition capable of inducing a specific CD4 and/or CD8 T-cell response.
